(19) United States
(12) Patent Application Publication
(10) Pub. No.: US 2012/0238660 A1
(43) Pub. Date: Sep. 20, 2012

(54) METHOD FOR DYEING A PLASTIC SUBSTRATE OF HIGH REFRACTIVE INDEX AND SUBSTRATE OBTAINED BY THIS METHOD

(75) Inventors: Yu Liu, Charenton-Le-Pont (FR); Lixin Song, Charenton-Le-Pont (FR); Yaw Chuan Lim, Charenton-Le-Pont (FR); Likui Wang, Charenton-Le-Pont (FR)

(73) Assignee: ESSILOR INTERNATIONAL (Compagnie Generale d'Optique), CHARENTON-LE-PONT (FR)

(21) Appl. No.: 13/508,757

(22) PCT Filed: Oct. 14, 2010
(86) PCT No.: PCT/IB2010/054662

§ 371 Date: May 9, 2012

(30) Foreign Application Priority Data
Nov. 10, 2009 (EP) 09306083.8

(51) Int. Cl. A61K 8/72; D06P 1/02; D06P 1/00

(52) U.S. Cl. 523/105; 8/506

(57) ABSTRACT

Method for dyeing a substrate prepared by curing a polymerizable composition having one monomer containing one episulfide group, the method including:
providing a dye solution including a dye and a carrier in a solvent, the carrier being chosen from the compounds represented by the following formulae (1) and (2):

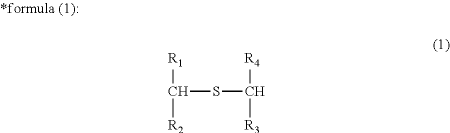

wherein R₁, R₂, R₃ and R₄ are each selected from the group consisting of alkyl having from 1 to 10 carbon atom, H, —CH₂SH, —CH₂SCH₂CH₂SH,

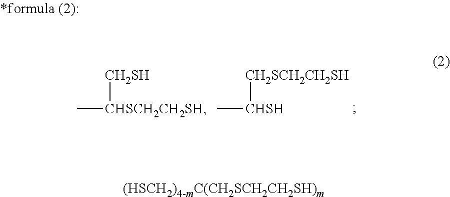

wherein m denotes an integer from 1 to 3;
and
at least partially immersing the substrate in the dye solution for a time sufficient to allow the effective tinting of the substrate.

The present invention pertains to a method for dyeing substrates to obtain tinted substrates or articles, for example tinted optical or ophthalmic lenses. The invention particularly concerns articles obtained from substrates prepared by curing a polymerizable composition comprising at least one monomer containing at least one episulfide group and optionally at least one thiol group.

The polymerization of said monomer leads to a polymer comprising at least one thioether group.

Substrate prepared by curing a polymerizable composition comprising at least one monomer containing at least one episulfide group, is herein after called “substrate of high refractive index comprising a thioether group”.

Tinting/colouring an ophthalmic lens is an increasing need.

However, substrates of high refractive index comprising a thioether group are difficult to tint with conventional processes such as conventional bath direct tinting. Water bath dyeing is not suitable because of its limited dye absorption amount, even during prolonged time.

Therefore there is a need for a process for tinting substrates of high refractive index comprising a thioether group, overcoming the limitations of the known processes, without compromising the tinting homogeneity. Advantageously the present invention discloses a method that addresses the issues discussed above.

The invention provides a solution for tinting substrates of high refractive index comprising a thioether group in a short time and achieving a higher tinting intensity with good uniformity. These and other advantages are achieved in accordance with the present invention as described below.

The invention discloses a method for dyeing a substrate prepared by curing a polymerizable composition comprising at least one monomer containing at least one episulfide group and optionally at least one thiol group, said method comprising the following successive steps:

providing a dye solution comprising at least one dye, or dye precursor, and at least one carrier in at least one solvent, the carrier being chosen from the compounds represented by the following formulae (1) and (2):

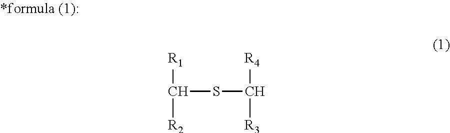

wherein R₁, R₂, R₃ and R₄ are each selected from the group consisting of the following radicals: alkyl having from 1 to 10 carbon atom, H, —CH₂SH, —CH₂SCH₂CH₂SH,

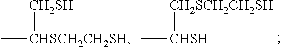

provided that R₁, R₂, R₃ and R₄ are not H or an alkyl simultaneously, or that if any three of R₁, R₂, R₃ and R₄ are H or an alkyl, the other radical consists of one of the following radicals:

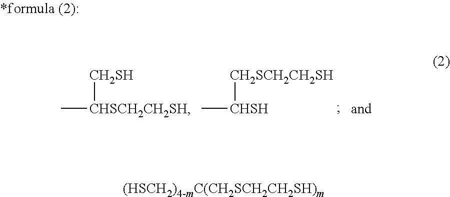

wherein m denotes an integer from 1 to 3;

And

at least partially immersing the substrate in the dye solution for a time sufficient to allow the effective tinting of the substrate.

According to the invention, the immersion step can be carried out once, or can be repeated several times, in order to manufacture a tinted substrate of higher intensity. This step can be carried out by the use of a dyeing apparatus, in which the substrate is moved in the dye solution according to scheduled sequences.

The substrate can be immersed totally, or partially. If the substrate is immersed partially, generally only the immersed part of the substrate is tinted.

The substrate can be a commercially available product, or it can be manufactured just before the tinting.

The substrate has generally the shape of an article.

The method according to the invention is rapid, reproducible and easy to carry out. Moreover the tinting of the part of the substrate which is immersed is substantially uniform.

In addition, this method allows the production of the whole range of coloration, from the lightest colour to the darkest colour.

Substrate, in the sense of the present invention, should be understood to mean an uncoated substrate. The substrate may in particular be an optically transparent material having the shape of an optical article.

According to a preferred embodiment, the substrate has a refractive index of at least 1.67, preferably from 1.67 to 1.79 and more preferably from 1.71 to 1.79.

The monomer containing at least one episulfide group and optionally at least one thiol group according to the invention contains episulfide group(s), or episulfide group(s) and thiol group(s).

In a preferred embodiment, the monomer does not comprise any other functional group than episulfide group(s) (and optionally thiol group(s)).

Preferably the episulfide group has the structure of formula (3):

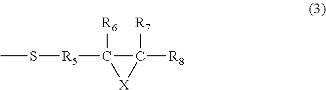

wherein R₅ represents a hydrocarbon group having 1 to 10 carbon atoms, R₆, R₇ and R₈ each represents a hydrogen atom or a hydrocarbon group having 1 to 10 carbon atoms, X represents S or O with the proviso that, in the molecule, the average number of S represented by X is about 50% of the total number of S and O in the three-member ring,

and more preferably X is S.

Preferably, the episulfide group has the structure of formula (4):

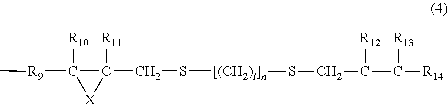

wherein R₉, R₁₀, R₁₁, R₁₂, R₁₃ and R₁₄ each represents a hydrogen atom or a hydrocarbon group having 1 to 10 carbon atoms, X represents S or O with the proviso that, in the molecule, the average number of S represented by X is about 50% of the total number of S and O in the three-member ring, m represents an integer from 0 to 6 and n represents an integer from 0 to 4, and more preferably X is S.

Preferably, the polymerizable composition further comprises one or more polythiols of formula R(SH)_{n′}, in which n′ is an integer from 2 to 6 and R′ is an organic group of valency equal to n′.

In a preferred manner the carrier is chosen from the group comprising straight-chain dimercaptan compounds such as bis(2-mercaptoethyl) sulfide, 1,2-[bis(2-mercaptoethylthio)]ethane; branched aliphatic polymercaptan compounds such as 2-mercaptomethyl-1,3-dimercaptopropane, 2-mercaptomethyl-1,4-dimercaptobutane, 2-(2-mercaptoethylthio)1,3-dimercaptopropane, 1,2-bis[(2-mercaptoethylthio)]-3-mercaptopropane, 1,1,1-tris(mercaptomethyl)propane and tetrakismercaptomethylmethane; 4-mercaptomethyl-3,6-dithia-1,8-octanedithiol and 2-mercaptoethyl sulphide, preferably said carrier being mercaptomethyl-3,6-dithia-1,8-octanedithiol or 2-mercaptoethyl sulphide, and more preferably the carrier being 4-mercaptomethyl-3,6-dithia-1,8-octanedithiol.

Preferably, the solvent is water or an organic solvent, and more preferably the solvent is water.

According to the invention, the dye solution can contain a dye chosen from the group of azo type dyes, quinophtalones type dyes, and anthraquinone type dyes.

However various known dyes mixture or dyes solutions used in dyeing can be used, as known to the one skilled in the art.

The dye solutions which are suitable are commercially available disperse dye solutions, to be combined with at least one carrier according to the invention. For example, the dyes sold as BPI® Molecular Catalytic Tints by

Brain Power International are available in more than 180 colours.

In a variant, the dye solution contains 0.05% to 5%, preferably 0.05% to 1%, and more preferably 0.08 to 0.5% by weight of the carrier.

The amount of the dye is not critical but is ordinarily 0.5 to 30 g/Liter in terms of concentration in the dye solution.

The dye solution can comprise any other additive known to the one skilled in the art. According to the invention, the dye solution can contain an emulsifier, preferably chosen from the group of amine salts or alkali salts of carboxylic, sulfamic or phosphoric acids, acid salts of amines, ethoxylated or propoxylated alkyl or aryl phenolic compounds.

The optional emulsifier (surfactant) may be used in an amount of 0 to 15 pbw, preferably 0.5 to 5 pbw, more preferably 3 to 4 pbw.

Emulsifiers which may be used include ionic, non-ionic, or mixtures thereof. Typical ionic emulsifiers are anionic, including amine salts or alkali salts of carboxylic, sulfamic or phosphoric acids, for example sodium lauryl sulfate, ammonium lauryl sulfate, lignosulfonic acid salts, ethylene diamine tetra acetic acid (EDTA), sodium salts and acid salts of amines such as laurylamine, hydrochloride or poly(oxy-1,2-ethanediyl), alpha-sulfo-omega-hydroxy ether with phenol 1-(methylphenyl)ethyl derivative ammonium salts; or amphoteric, that is compounds bearing both anionic and cationic groups, for example lauryl sulfobetaine dihydroxy ethylalkyl betaine; amido betaine based on coconut acids; disodium N-lauryl amino propionate; or the sodium salts of dicarboxylic acid coconut derivatives. Typical non-ionic emulsifiers include ethoxylated or propoxylated alkyl or aryl phenolic compounds such as octylphenoxypolyethyleneoxyethanol or poly(oxy-1,2-ethanediyl), alpha-phenyl-omega-hydroxy, styrenated. The preferred emulsifier is a mixture of C₁₄-C₁₈ and C₁₆-C₁₈ ethoxylated unsaturated fatty acids and poly(oxy-1,2-ethanediyl), alpha-sulfo-omega-hydroxy ether with phenol 1-(methylphenyl) ethyl derivative ammonium salts and poly(oxy-1,2-ethanediyl), alpha-phenyl-omega-hydroxy, styrenated.

The method of the invention provides tinted substrates in a short time and achieves a higher tinting intensity with good uniformity.

The method of the invention enables tinting substrates to be provided of different luminous transmittance depending mainly on different tinting times and dye solutions of different dye concentration.

Preferably, the length of the immersion is within the range of from 3 to 100 minutes, preferably from 10 to 90 minutes, and more preferably from 30 to 96 minutes.

According to the invention, the method can further comprise the heating of the dye solution at a temperature of from 90 to 100° C., preferably from 90 to 96° C., and more preferably from 93 to 96° C., during the immersion.

The invention also concerns an article, preferably an ophthalmic lens, comprising a tinted substrate which is obtained by the method described above.

According to the invention, the article is preferably such that the luminous transmittance of the article is lower than 20%, preferably lower than 15%, and more preferably lower than 10%.

Said article is advantageously coated with coatings conventionally used in the ophthalmic optics field, such as anti abrasion coatings, scratch resistant coatings or anti-reflection coatings.

The invention thus also concerns said article further comprising at least partially a coating.

The invention will be further illustrated by the following non-limiting examples which are given for illustrative purposes only.

### EXAMPLES

In all the examples, the substrate is a commercially available compound provided by the company Mitsubishi Gas Chemical and sold as having a refractive index n_{D} of 1.74.

This substrate has a density d of 1.45, and was obtained from the chemical curing of 2-mercaptoethyl sulfide

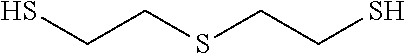

(and metallic sulphur S_{α} 20% by weight),

and

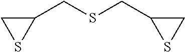

2,3-episulfide propanyl sulfide.

### Example 1

Two compounds, 4-mercaptomethyl-3,6-dithia-1,8-octanedithiol and 2-mercaptoethyl sulfide, were used as a carrier for tinting two substrates, in two different water bath solutions, according to the conditions set forth in the Table below. Substrates as disclosed above were immersed in respective bath. The concentration of the carrier was 0.1 vol. % in each bath solution.

Each water bath solution comprises the carrier and a dye solution (which is a commercially available disperse dye formulation sold as BPI dye solution #46300) diluted in water.

The two tinted substrates so obtained are two ophthalmic lenses according to the invention.

The results of the measurements on said ophthalmic lenses are given in the Table below, in which Tv is the luminous transmittance and Hz is the haze.

| | | | | Homogeneity |
|---|---|---|---|---|
| | | | | of the tinting |
| | Tv | Hz | Conditions | (visual) |
| 4-mercaptomethyl- | 32.5 | 0.45 | Ambient | Pass |
| 3,6-dithia-1,8- | | | pressure | |
| octanedithiol | | | 94° C. | |
| | | | 45 min | |
| 2-Mercaptoethyl | 3.78 | 0.93 | Ambient | Pass |
| Sulfide | | | pressure | |
| | | | 94° C. | |
| | | | 1 hr | |

Good quality tinting was obtained, and the haze level of the ophthalmic lenses using those two different carriers was acceptable. The effect of 4-mercaptomethyl-3,6-dithia-1,8-octanedithiol was better in terms of haze level.

The haze value of the final ophthalmic lenses was measured by light transmission using the Haze-Guard Plus haze meter from BYK-Gardner (a color difference meter) according to the method of ASTM D1003-00, which is incorporated herein in its entirety by reference. All references to “haze” values in this application are by this standard. The instrument was first calibrated according to the manufacturer's instructions. Next, the sample was placed on the transmission light beam of the pre-calibrated meter and the haze value was recorded from three different specimen locations and averaged. Luminous transmittance (also called “relative light transmission factor in the visible spectrum”) Tv (or ζv) is defined in the standard ISO 13666:1998 and is measured according to the standard ISO 8980-3 (from 380 to 780 nm), using the same device.

### Example 2

Four pieces of the substrate disclosed above were tinted into lenses respectively for 15, 30, 45, 60 minutes in four different bath solutions. Each water bath solution comprises the carrier 4-mercaptomethyl-3,6-dithia-1,8-octanedithiol, under 0.1% by volume concentration, and a dye solution (which is a commercially available disperse dye formulation sold as BPI lens dye solution #46300) diluted in water.

The temperature during the immersion was equal to 94° C.

The results of the measurements of the luminous transmittance (T) and of the haze (Hz) on the four substrates respectively on the so obtained four ophthalmic lenses are given in the Table below, before respectively after the tinting.

Transmission, haze vs Tinting Time

| | |
|---|---|
| Before | After |

| Time (min) | Tv | Hz | Tv | Hz |
|---|---|---|---|---|
| 0 | | | 87 | |
| 15 | 87.0 | 0.37 | 72.5 | 0.38 |
| 30 | 87.0 | 0.38 | 48.7 | 0.42 |
| 45 | 87.1 | 0.32 | 32.5 | 0.47 |
| 60 | 87.1 | 0.39 | 12.3 | 0.51 |

The transmission of the so obtained ophthalmic lens may reach class 3 after 60 minutes immersion in the bath under 94° C. The haze level does not exhibit significant change before and after tinting.

Good quality tinting was obtained, and the haze level of the ophthalmic lenses was acceptable. This example shows the possibility of varying the luminous transmittance of the ophthalmic lenses by adjusting tinting time.

1. Method for dyeing a substrate prepared by curing a polymerizable composition comprising at least one monomer containing at least one episulfide group and optionally at least one thiol group, said method comprising the following successive steps:
providing a dye solution comprising at least one dye, or dye precursor, and at least one carrier in at least one solvent, the carrier being chosen from the compounds represented by the following formulae (1) and (2): 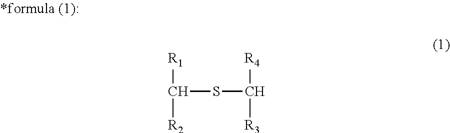
wherein R₁, R₂, R₃ and R₄ are each selected from the group consisting of the following radicals: alkyl having from 1 to 10 carbon atom, H, —CH₂SH, —CH₂SCH₂CH₂SH, 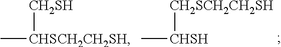
provided that R₁, R₂, R₃ and R₄ are not H or an alkyl simultaneously, or that if any three of R₁, R₂, R₃ and R₄ are H or an alkyl, the other radical consists of one of the following radicals: 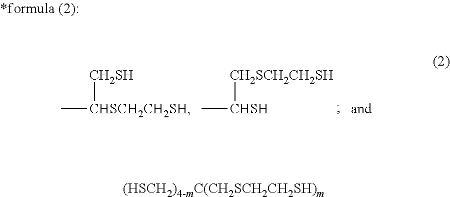
wherein m denotes an integer from 1 to 3;
And
at least partially immersing the substrate in the dye solution for a time sufficient to allow the effective tinting of the substrate.

2. Method according to claim 1, wherein the substrate has a refractive index of at least 1.67, preferably from 1.67 to 1.79 and more preferably from 1.71 to 1.79.

3. Method according to claim 1, wherein the episulfide group has the structure of formula (3): 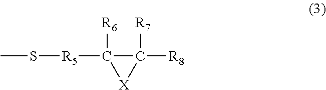
wherein R₅ represents a hydrocarbon group having 1 to 10 carbon atoms, R₆, R₇ and R₈ each represents a hydrogen atom or a hydrocarbon group having 1 to 10 carbon atoms, X represents S or O with the proviso that, in the molecule, the average number of S represented by X is about 50% of the total number of S and O constituting in the three-member ring,
and preferably X is S.

4. Method according to claim 1, wherein the episulfide group has the structure of formula (4): 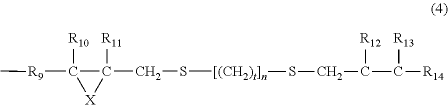
wherein R₉, R₁₀, R₁₁, R₁₂, R₁₃ and R₁₄ each represents a hydrogen atom or a hydrocarbon group having 1 to 10 carbon atoms, X represents S or O with the proviso that, in the molecule, the average number of S represented by X is about 50% of the total number of S and O in the three-member ring, m represents an integer from 0 to 6 and n represents an integer from 0 to 4, and preferably X is S.

5. Method according to claim 1, wherein the polymerizable composition further comprises one or more polythiols of formula R′(SH)_{n′}, in which n′ is an integer from 2 to 6 and R′ is an organic group of valency equal to n′.

6. Method according to claim 1, wherein said carrier is chosen from the group comprising straight-chain dimercaptan compounds such as bis(2-mercaptoethyl) sulfide, 1,2-[bis(2-mercaptoethylthio)]ethane; branched aliphatic polymercaptan compounds such as 2-mercaptomethyl-1,3-dimercaptopropane, 2-mercaptomethyl-1,4-dimercaptobutane, 2-(2-mercaptoethylthio)1,3-dimercaptopropane, 1,2-bis[(2-mercaptoethylthio)]-3-mercaptopropane, 1,1,1-tris(mercaptomethyl)propane and tetrakismercaptomethylmethane; 4-mercaptomethyl-3,6-dithia-1,8-octanedithiol and 2-mercaptoethyl sulphide, preferably said carrier being mercaptomethyl-3,6-dithia-1,8-octanedithiol or 2-mercaptoethyl sulphide, and more preferably the carrier being 4-mercaptomethyl-3,6-dithia-1,8-octanedithiol.

7. Method according to claim 1, wherein the solvent is water or an organic solvent, and preferably the solvent is water.

8. Method according to claim 1, wherein the dye solution contains a dye chosen from the group of azo type dyes, quinophtalones type dyes, and anthraquinone type dyes.

9. Method according to claim 1, wherein the dye solution contains 0.05% to 5%, preferably 0.05% to 1%, and more preferably 0.08 to 0.5% by weight of the carrier.

10. Method according to claim 1, wherein the dye solution contains an emulsifier, preferably chosen from the group of amine salts or alkali salts of carboxylic, sulfamic or phosphoric acids, acid salts of amines, ethoxylated or propoxylated alkyl or aryl phenolic compounds.

11. Method according to claim 1, wherein the length of the immersion is within the range of from 3 to 100 minutes, preferably from 10 to 90 minutes, and more preferably from 30 to 96 minutes.

12. Method according to claim 1, wherein the method further comprises the heating of the dye solution at a temperature of from 90 to 100° C., preferably from 90 to 96° C., and more preferably from 93 to 96° C., during the immersion.

13. Article, preferably an ophthalmic lens, comprising a tinted substrate which is obtained by the method of claim 1.

14. Article according to claim 13, wherein the luminous transmittance of the article is lower than 20%, preferably lower than 15%, and more preferably lower than 10%.

15. Article according to claim 13, wherein said article further comprises at least partially a coating.
